(19) United States
(12) Patent Application Publication
(10) Pub. No.: US 2017/0262589 A1
(43) Pub. Date: Sep. 14, 2017

(54) SYSTEMS AND METHODS FOR PHYSICALLY SUPPORTING USERS DURING EXERCISE WHILE ENHANCED OXYGEN TREATMENT

(71) Applicant: Mesa Digital, LLC, Albuquerque, NM (US)

(72) Inventor: Luis M. Ortiz, Albuquerque, NM (US)

(21) Appl. No.: 15/455,592

(22) Filed: Mar. 10, 2017

Related U.S. Application Data
(60) Provisional application No. 62/308,399, filed on Mar. 15, 2016.
(60) Provisional application No. 62/307,733, filed on Mar. 14, 2016.

(51) Int. Cl. G06F 19/00; A61H 3/00; A61G 5/10; A61B 5/00; A61B 5/117

(52) U.S. Cl. CPC G06F 19/325; G06F 19/3481; G06F 19/322; A61B 5/4848; A61B 5/117; A61B 5/0022; A61B 5/6802; A61G 5/1091; A61H 3/008; H04L 63/08

(57) ABSTRACT

User access to exercise with oxygen therapy (EWOT), therapy progress data, and user profile/record integration can be enabled via portable and wearable computing devices in use at treatment facilities is described. Embodiments leverage the field of wireless communications can access facility-based transponders, user authentication, data access, and data tracking. The invention enables access to oxygen therapy during exercise at various facilities by authorizing user access, tracking usage, providing access to user profiles/records, recording user progress, providing notification to providers of subsequent user treatment, and user record management based on proximity of a provider to a user during treatment. A safety harness system can include a circular padded rail to encircle the waist/torso of a user underneath the user's arms; hydraulically controlled legs can enable manipulation/movement under hydraulic control when the padded rail is secured to the user and while the user is moving on exercise equipment.

### PRIORITY

The disclosed embodiments claim priority to U.S. Provisional Patent Application No. 62/308,399, filed Mar. 15, 2016, entitled “SYSTEMS AND METHODS FOR PHYSICALLY SUPPORTING USERS DURING EXERCISE WHILE ENHANCED OXYGEN TREATMENT,” and to U.S. Provisional Patent Application No. 62/307,733, filed Mar. 14, 2016, entitled “SYSTEMS AND METHODS FOR ACCESSING AND MANAGING ENHANCED OXYGEN TREATMENT DURING EXERCISE,” which are both herein incorporated by reference.

### TECHNICAL FIELD

The disclosed embodiments are generally related to physical fitness, medical/healthcare management, exercise with oxygen therapy (EWOT), and its use in healthcare and fitness. Embodiments are additionally related to providing physical support to therapy patients as users of EWOT when using exercise equipment in order to gain the maximum benefit from EWOT.

### BACKGROUND OF THE INVENTION

The following published United States patent applications are incorporated by reference herein in their entirety for their teachings, not because they specifically teach the patent eligible aspects of the present invention as ultimately claimed by the inventors, but because the references provide technical background: U.S. Pat. No. 8,375,938, U.S. Pat. No. 8,616,200, U.S. Pat. No. 7,141,029, US Publication 20140142396; US Publication US Publication 20140143064; US Publication 20140258208; US Publication 20140297642; US Publication 20140361147; US Publication 20150003214; US Publication 20150005912.

Hyperbaric and hypoxic chamber systems are known and used in the medical and sports industries. In essence, occupants of hyperbaric chambers undergo hyperbaric treatments in which they are subjected to relatively high pressures of oxygen. Hyperbaric treatments are known, amongst other things, to enhance muscular recuperation, increase oxygen inhalation, etc. In hypoxic chambers, the occupant is subjected to lower oxygen contents to simulate high altitudes. Hypoxic treatments are known, amongst other things, to stimulate the production of red blood cells.

There is a movement to integrate oxygen and exercise. It is not practical to construct large chambers that can house exercise equipment and occupants during exercise. Because of space limitations, systems have been invented that enables users to wear an oxygen mask while exercising. EWOT (abbreviation for “exercise with oxygen therapy”) is the term coined to refer to the concept of incorporation oxygen via masks with exercise. Oxygen concentrators are operating in doctors' offices, clinics, rehabilitation facilities, and in private settings for people who want to experience the benefits of exercising with oxygen therapy. As shown in FIG. 1 labeled prior art, a typical EWOT system can include a 5 or 10 LPM Oxygen Concentrator 101 connected to a non-rebreather mask 105. The optimal mask 105 for EWOT is preferably worn on the face, covering the nose and mouth, which excludes room air and permits only oxygen rich (or oxygen depleted) air to be consumed by the wearer. A humidifier bottle 103 can be provided inline with tubing connecting the concentrator 101 with non-rebreather masks 105 in order to introduce relieving moisture into the enhanced oxygen provided from the oxygen concentrator. Additionally, a reservoir 106 can be incorporated into the system in order to enhance concentrated oxygen delivery volume beyond what can normally be accomplished by merely using a concentrator 101. Whole Health Network provides the reservoir 106 as a hanging/supported bag system, pictured in FIG. 1, which is a product referred to as LiveO2. A reservoir is reported to provide enough enhanced oxygen for a user to draw from for a fifteen-minute workout. The Whole Health Network reservoir is provided as a semi-permeable, inert fabric container, which stores oxygen produced by an air separator.

Exercise equipment 107 can take many forms, but is typically limited to stationary systems because it must be located next to currently available EWOT systems. Such exercise equipment can include stationary bicycles, treadmills, elliptical machines, climbing machines, skiing machines, at the like. There is also specialized equipment that has been developed that enhances body oxygenation. For example, Super EWOT is the process of breathing high concentrations of oxygen while standing on “sonic wave” vibration systems, which have been shown to produce a negative polarity directly into the human body.

U.S. Pat. No. 7,141,029, issued to Kim, describes a sonic vibration system currently marketed by Turbosonic USA. Reference numeral 107 in FIG. 1 is a picture of a sonic wave vibration system representing exercise equipment 107. According to Turbosonic USA, disease cannot survive in highly oxygenated bodies. Using body vibration therapy three times a week while breathing higher concentrations of oxygen can increase the body's oxygen uptake and gives the cells the oxygen molecules required to process ATP, which can provide a person with more energy. Oxygen uptake has been shown to be greatly enhanced during sonic vibration due to the cell charging that occurs during the magnetic resonating field of negative polarity. Human bodies are bio-electric batteries, and when a body's cell voltage drops a person can become sick. Cell voltage can be increased by using a sonic vibration system (such as all Turbo Sonic vibration systems or the Sonic Life) while engaging in deep breathing. There is documentation that reveals how blood is affected by oxygenation as a result of EWOT after using a sonic vibration system. When cells become charged and then repel each other, greater oxygen uptake, cellular communication, and cellular waste removal can be achieved.

Exercise equipment can pose some danger or hazard to a user, depending on the user. For example, it may be easy for most users to mount and manipulate an exercise bike, treadmill, or ski machine, but this would be very challenging for Lou Gehrig's disease (Amyotrophic lateral sclerosis-ALS), multiple sclerosis (MS), and stroke patients as users (just to name a few conditions). Such a user requires hands on assistance by therapists so that the user does not fall or have an accident. This limits the type of equipment that such a user can use to engage in EWOT to its maximum benefit. The sonic vibration system mentioned above (U.S. Pat. No. 7,141,029) is perhaps the least risky for less mobile, recovering user to use; however, it is still an open system having only a handle bar system for support of the patient/user. The sonic vibration system will require hands on therapists support depending on the condition of the patient/user.

In order to obtain the benefits of EWOT, it has been recommended that a person should exercise while breathing at least 8 LPM (Liters Per Minute) of 90-95% pure oxygen. The reported and documented benefits of EWOT include inter alia the following:

| | |
|---|---|
| Stimulate brain activity | Build endurance |
| Increase memory capacity | Detox your blood |
| Boost concentration | Reduce stress |
| Develop stronger alertness | Calm anxiety |
| Raise energy levels | Alleviate tension headaches |
| Improve strength | Help with cardiovascular activity |
| Remedy irregular sleeping patterns | Strengthen the immune system |
| Prevent lactic add build up | |

Wearable computing devices (“wearable devices”) come in a variety of implementations and configurations. For example, some wearable computing devices are implemented in the context of wristwatch type devices capable of wireless data communication with neighboring devices (e.g., smartphones) or remote devices (e.g., servers) the Internet. Fitness wristbands are in use to track user activity and provide feedback data to users. Smart watches are now available that provide some functionality of smartphones and tablets, but in a smaller user interface wearable on a user's wrist. Additional activity and medical monitoring devices can be mounted on a user's body and can be used to monitor/track users and assist with treating medical conditions, such as monitors used to measure insulin in the treatment of diabetes and sensors for reading vital signs (e.g., heart rate).

Some of the main features of wearable computers are in the ability for them to achieve and maintain data currency and consistency. Wearable devices can also provide user access to data from systems they connect with. There is a constant interaction between the portable, wearable computer and its user, i.e., there is no need to turn the device on or off. Another feature is the ability to multi-task. It is not necessary to stop what you are doing to use the device; it is augmented into all other actions. These computer devices can be incorporated by the user to act like a prosthetic. It can therefore be like an extension of the user's mind and/or body.

Electronic records (ER) associated with healthcare include Electronic medical records (EMR), electronic health records (EHR), and is being expanded to include therapeutic exercise records. ERs continue to displace manual record keeping because of the ubiquity of electronic data storage solutions that are more accessible from remote servers, yet can be secured. ERs are significantly growing in use outside of healthcare facilities given the need to monitor the healthcare and progress of released and home-based patients. Despite the growing ubiquity of ERs for recording action on or in association with a user, there is still a growing need to assure security, provider access to timely information, and access control.

In light of the foregoing background, what are needed are systems and methods to provide expanded access to exercise with enhanced oxygen therapy during physical fitness. What is also needed are methods and system for providing and managing user access to EWOT in diverse facilities, providing support for challenged users with motion limiting conditions, for tracking user therapy, maintaining user records and profiles associated with therapy, and ensuring the safety of therapy of EWOT facility users. These and other unique enhancements for EWOT are further described herein.

### BRIEF SUMMARY

The following summary is provided to facilitate an understanding of some of the innovative features unique to the disclosed embodiment and is not intended to be a full description. A full appreciation of the various aspects of the embodiments disclosed herein can be gained by taking the entire specification, claims, drawings, and abstract as a whole.

It is therefore an aspect of the disclosed embodiments to provide, inter alia, systems and methods for any of: providing user access to exercise with oxygen (EWOT) systems at various facilities, provide for user safety when using exercise equipment while engaging in EWOT therapy, for the management of EWOT access and usage, tracking of user EWOT activity, and integration of user EWOT activity with user profiles (e.g., health records).

It is therefore an aspect of the disclosed embodiments that a user of an EWOT system can be granted access to its use via at least one of: user biometrics, passcodes, smart cards, or a portable electronic wireless devices (e.g., smartphone, wearable wrist device). A user interface associated with an EWOT system and exercise equipment can enable interaction with the user via user input or wirelessly via the user's portable device. The user interface can include a payment mechanism for occasional users that are not concerned with recording data and updated profiles, but would rather pay for use with a credit card or mobile payment application from a portable device (e.g., smartphone, smart watch).

It is yet another feature that a wearable device be used to enable at least one of: obtain access to an EWOT system, track user activity, and integrate with a user profile to update the profile with tracked EWOT activity. A wrist beacon can be provided as a wearable device on users utilizing treatment facilities (e.g., gyms, health clubs, resorts, spas, sports training facilities). The wrist beacon can provide a unique user identification code, maintain access to an EWOT system and/or facility wireless network, enable user location determination, and enable recording of activity with health records stored in a remote server.

It is another aspect of the disclosed embodiments to enable authentication of users when in close proximity to an EWOT system. Proximity can be established via wireless signals (e.g., Bluetooth, NFC, RFID, Wi-Fi) from any combination of wearable device, portable computers (e.g., tablets, smartphones), and can include the use of biometrics upon initiation of treatment or records access requests. Biometrics can be provided in the form of voice, fingerprint and eye scan, or by user entry of exercise machine identification information. User authentication and location information can be used as data for tracking fitness, environment, privileges, and for use in recording therapy.

It is yet another aspect of the disclosed embodiments to provide for a wearable devices that can be integrated as beacons or with beacons (e.g., transponders) within a health care facility to associate EWOT system users with EWOT equipment and/or facilities, enable record interactions between record custodians and users, and provide and control access to EWOT systems and records. With such a system, services are capable of being wirelessly communicated between a wearable device and at least one transponder out of a plurality of transponders and dispersed throughout a facility in association with associated EWOT systems and workout equipment. The at least one transponder can be within at least a Bluetooth range or a Wi-Fi range of communication with said wearable device. The location of a wearable device can be determined via said at least one transponder and based on a proximity of said wearable device to said at least one transponder.

It is yet another aspect of the disclosed embodiments to provide for a wearable device that can be wirelessly read by a nearby sensor and/or access a data network and determine a user's location (e.g., user location within a treatment room simultaneously with doctor's location within same treatment room).

It is also an aspect of the disclosed embodiments to provide security over data communicated with a wearable device. A system and method can provide an authorized user with data based on the user's identity and location as determined by any of a wearable device or portable computer.

A wireless communications module can be integrated in or associated with the wearable device to enable any: tracking, detecting, and communications with networks and transponders as needed to access data and manage data.

It still another aspect of the disclosed embodiments to provide for a method of determining the location of a user within a facility using radio frequency transponders in near communication (e.g., Bluetooth, Wi-Fi, RFID, NFC, etc.) with a wearable device, and authenticating the healthcare provider/user via RF signatures and/or biometric attributes.

It is another feature of the present invention that authentication can be facilitated by, for example, a remote server. The data and/or the services accessed based on the identity of the user can be retrieved from a remote server.

It is yet another feature of the present invention that the wearable device can be associated with a wireless hand held communications device. The data and/or the services can be wirelessly communicated between the wearable device and the wireless hand held communications device (e.g., via Bluetooth communications, RFID, NFC, WiFi, etc.). The wireless hand held communications device can be authenticated based on, for example, the at least one biometric. Additionally, data and/or services can be wirelessly communicated between the wearable device and at least one transponder out of a plurality of transponders dispersed throughout a facility. In general, the at least one transponder may be within at least NFC (near field communications) range and up to RFID, Bluetooth, or Wi-Fi range communication with the wearable device.

It is also a feature to provide updated data that can be wirelessly delivered and/or wirelessly provided to the wearable device with respect to the at least one transponder based on the authenticating the user via the wearable device. The data can be, for example, past medical treatments (e.g., past surgeries, therapies, prescriptions), current medical treatments (e.g., current prescriptions), future medical treatments (scheduled procedures), and health maintenance information (e.g., fitness history/progress).

In yet another feature, a user profile can be established with respect to a user authenticated by a system to establish an access level with respect to the user for access to the data and/or EWOT services.

It is yet another feature of the present invention that healthcare providers, such as primary physicians, can be automatically authorized in a user's profile to be automatically notified when/if a user associated with the healthcare provider receives treatment (emergency or otherwise) by another or subsequent service provider, at a different or new facility, while on travel, or otherwise, when subsequent treatment is stored in a server containing the user's records, and the notification can occur electronically via known communication methods (e.g., text, email, automated voice messaging).

It is yet another feature of the present invention that a user can authorize which healthcare providers are provided access to records, and/or are provided with automated messages of any treatment received by a user by other associated providers, new providers, facilities, whether treatment is elective or in association with emergency circumstances.

It is yet another feature that the system can synchronize recording of treatment provided to the user and the identity of providers providing therapy to the user between the at least one server and mobile computing devices associated with the user or therapist. Updates of recorded treatment from the at least one server can occur as a push notification to computers and mobile computing devices associated with the healthcare providers.

It is yet another aspect of the disclosed embodiments to provide a safety harness system to support users while engaged in exercise on exercise equipment associated with EWOT.

It is also an aspect that the harness system be capable of encircling the user under the user's shoulders with a padded rail and providing hydraulically controlled support arms to enable a user to squat or bend while engaging in exercise.

It is yet another aspect that the harness system be capable of coupling with existing exercise equipment (e.g., sonic therapy platforms) while encircling the user under the user's shoulders with a padded rail and providing hydraulically controlled support arms to enable a user to squat or bend while engaging in exercise.

It is yet another aspect of the disclosed embodiments that a user of an EWOT system can be granted access to its use via at least one of: user biometrics, passcodes, smart cards, or a portable electronic wireless devices (e.g., smartphone, wearable wrist device). A user interface associated with EWOT system and exercise equipment can enable interaction with the user via user input or wirelessly via the user's portable device.

It is yet another feature that a wearable device be used to enable at least one of: obtain access to an EWOT system, track user activity from sensor associated with any of EWOT systems, the support harness and exercise equipment, and integrate with a user profile to update the profile with tracked EWOT activity. A wrist beacon can be provided as a wearable device on users utilizing treatment facilities (e.g., hospitals, therapy clinics, gyms, health clubs, resorts, spas, sports training facilities). The wrist beacon can provide a unique user identification code, maintain access to an EWOT system and/or facility wireless network, enable user location determination, and enable recording of activity with health records stored in a remote server.

A wireless communications module can be integrated in or associated with the wearable device to enable any: tracking, detecting, and communications with networks and transponders as needed to access data and manage data.

It is still another aspect of the disclosed embodiments to provide for a method of determining the location of a user within a facility using radio frequency transponders in near communication (e.g., Bluetooth, Wi-Fi, RFID, NFC, etc.) with a wearable device, and authenticating the healthcare provider/user via RF signatures and/or biometric attributes.

It is another feature of the present invention that authentication can be facilitated by, for example, a remote server. The data and/or the services accessed based on the identity of the user can be retrieved from a remote server.

It is also a feature to provide updated data that can be wirelessly delivered and/or wirelessly provided to the wearable device with respect to the at least one transponder based on the authenticating the user via the wearable device. The data can be, for example, past medical treatments (e.g., past surgeries, therapies, prescriptions), current medical treatments (e.g., current prescriptions), future medical treatments (scheduled procedures), and health maintenance information (e.g., fitness history/progress).

In yet another feature, a user profile can be established with respect to a user authenticated by a system to establish an access level with respect to the user for access to the data and/or EWOT services.

It is yet another feature of the present invention that healthcare providers, such as primary physicians, can be automatically authorized in a user's profile to be automatically notified when/if a user associated with the healthcare provider receives treatment (emergency or otherwise) by another or subsequent service provider, at a different or new facility, while on travel, or otherwise, when subsequent treatment is stored in a server containing the user's records, and the notification can occur electronically via known communication methods (e.g., text, email, automated voice messaging).

It is yet another feature of the present invention that a user can authorize which healthcare providers are provided access to records, and/or are provided with automated messages of any treatment received by a user by other associated providers, new providers, facilities, whether treatment is elective or in association with emergency circumstances.

It is yet another feature that the system can synchronize recording of treatment provided to the user and the identity of providers providing therapy to the user between the at least one server and mobile computing devices associated with the user or therapist. Updates of recorded treatment from the at least one server can occur as a push notification to computers and mobile computing devices associated with the healthcare providers.

### BRIEF DESCRIPTION OF THE DRAWINGS

The accompanying figures, in which like reference numerals refer to identical or functionally similar elements throughout the separate views and which are incorporated in and form a part of the specification, further illustrate the present invention and, together with the detailed description herein, serve to explain the principles of the disclosed embodiments.

FIG. 1 illustrates a system diagram of EWOT system related devices interacting in accordance with carrying out aspects of the present invention;

FIG. 2 illustrates a system diagram of portable devices interacting with an EWOT system in accordance with carrying out aspects of the present invention;

FIG. 3 illustrates features of a wearable computing device (e.g., wristband) and features that it can include in accordance with a feature of the present invention;

FIG. 4 illustrates a user profile that can be stored in a server and devices that can access the profile, and/or be associated with the profile, in accordance with features of the present invention;

FIG. 5 illustrates a system diagram of how a first service provider (e.g., primary) authorized in a user profile to receive notifications when the user receives therapy, can receive electronic notifications when the user receives subsequent therapy (whether elective or emergency) by another provider or facility after the user's profile is updated in a server via a data network;

FIG. 6 illustrates a system diagram for a facility wherein transponders and wearable computers can be in operation by users, EWOT providers to associated users and providers, and manage data associated with the user;

FIG. 7 illustrates a high-flow chart of operations depicting logical operational steps of a method for providing data and/or services to a user when a portable computer device worn by a user is used, in accordance an alternative embodiment;

FIG. 8 illustrates a high-flow chart depicting logical operational steps of a method for providing data and/or services to a provider when a wearable computer device worn by a user is used;

FIG. 9 illustrates a high-flow chart depicting logical operational steps of a method for providing data and/or services to a provider when a wearable computer device won by a user is used; and

FIG. 10 illustrates a diagram of a safety harness system that can be adapted for use with exercise equipment during EWOT therapy.

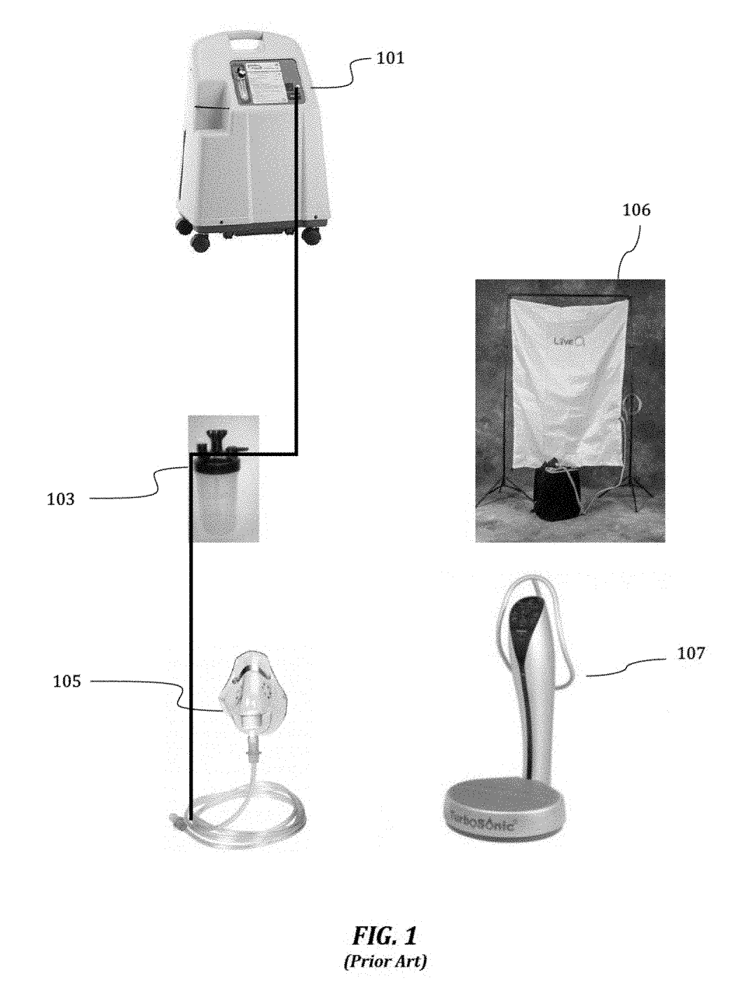

### DETAILED DESCRIPTION

The particular values and configurations discussed in these non-limiting examples can be varied and are cited merely to illustrate at least one embodiment and are not intended to limit the scope thereof.

The embodiments will now be described more fully hereinafter with reference to the accompanying drawings, in which illustrative are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the disclosed embodiments. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which disclosed embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As will be appreciated by one skilled in the art, the present invention can be embodied as a method, system, and/or a processor-readable medium. Accordingly, the embodiments may take the form of an entire hardware application, an entire software embodiment, or an embodiment combining software and hardware aspects all generally referred to herein as a “circuit” or “module.” Furthermore, the embodiments may take the form of a computer program product on a computer-usable storage medium having computer-usable program code embodied in the medium. Any suitable computer readable medium may be utilized including, for example, hard disks, USB Flash Drives, DVDs, CD-ROMs, optical storage devices, magnetic storage devices, etc.

Computer program code for carrying out operations of the disclosed embodiments may be written in an object oriented programming language (e.g., Python, Java, PHP C++, etc.) The computer program code, however, for carrying out operations of the disclosed embodiments may also be written in conventional procedural programming languages, such as the “C” programming language or in a visually oriented programming environment, such as, for example, Visual Basic.

The program code may execute entirely on the user's computer, partly on the user's computer, as a stand-alone software package, partly on the user's computer and partly on a remote computer, or entirely on the remote computer. In some described scenarios, the wearable device may be connected to sensor deployed throughout a facility and obtain communication to a network and remote server via NFC, RFID, Bluetooth, WiFi, and cellular data communications. In other scenarios, remote server and secured cloud-based storage facilities may be connected to a user's computer through a local area network (LAN) or a wide area network (WAN), wireless data network e.g., Wi-Fi, Wimax, 802.xx, Bluetooth and cellular data communications networks, or the connection may be made to an external computer via most third party supported networks (for example, through the Internet using an Internet Service Provider).

Aspects of the disclosed embodiments can be implemented as an “app” or application software that runs in, for example, a web browser and/or is created in a browser-supported programming language (e.g., such as a combination of JavaScript, HTML, and CSS) and relies on a web browser to render the application. The ability to update and maintain web applications without distributing and installing software on potentially thousands of client computers is a key reason for the popularity of such apps, as is the inherent support for cross-platform compatibility. Common web applications include webmail, online retail sales, online auctions, wikis and many other functions. Such an “app” can also be implemented as an Internet application that runs on smartphones, tablet computers, wearable devices, and other computing devices such as laptop and personal computers.

The disclosed embodiments are described in part below with reference to flowchart illustrations and/or block diagrams of methods, systems, and computer program products and data structures according to preferred and alternative embodiments. It will be understood that each block of the illustrations, and combinations of blocks, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

Reference is made to healthcare providers, facilities, patients, and users throughout this disclosure. It should be appreciated that healthcare providers can include fitness instructors, occupational therapists, physicians, nurses, and the like. Facilities can include healthcare facilities, hospitals, gyms, private clubs, spas, hotels, resorts, and the like. Users can be patients receiving therapy or simply individuals seeking better fitness outcomes. No limitation is meant by reference to a particular place, person, system, and/or event.

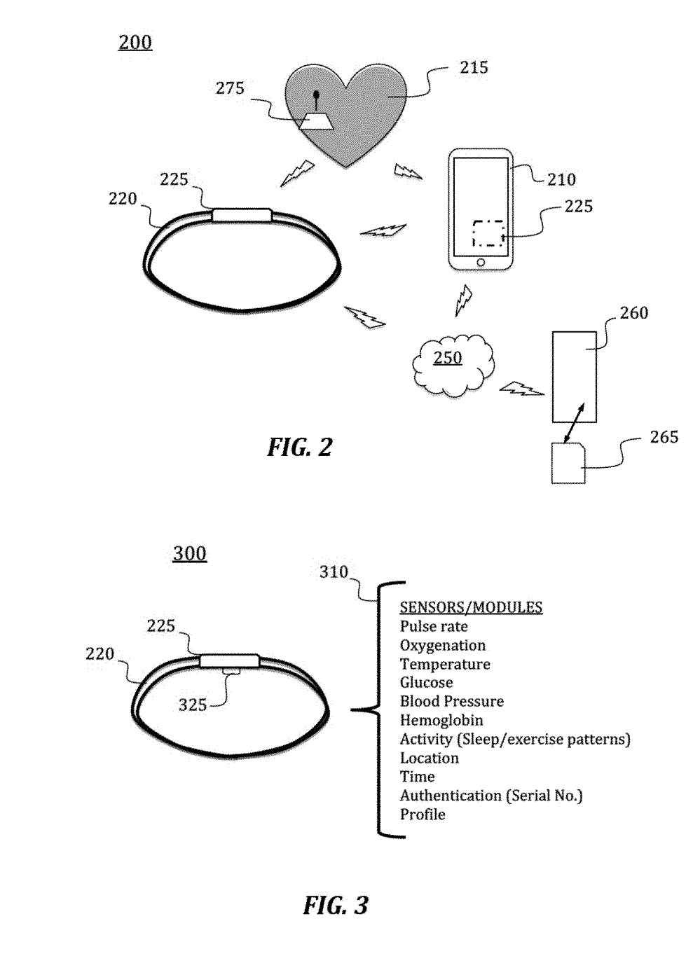

Referring to FIG. 2, a system diagram 200 illustrates aspects of the present invention wherein an exercise with oxygen (EWOT) system 215 can be associated with a user of a mobile device associated with a user in order to authorize user access to EWOT. The mobile device can be provided as part of a wearable device 220 (e.g., wristband as shown) including electronic circuitry 225 (including wireless communications) necessary to communicate with a transponder 275 associated with the EWOT system 215 and/or a mobile handheld device 210 (e.g., smartphone) carried by a user and also including the necessary electronic circuitry 225. Any type of computing device including a wireless transmitter (including smartphones, laptop computers, or tablet computing devices, etc.) can be configured to communicate data with a wearable computing device 210 worn by a user and/or the transponder 275. The mobile device 210 and the wearable computing device 220 can also contain Electronic circuitry 225 to enable either of them to establish a data communication link directly with the transponder 275, and can also communicate with a remote server 260 via a data communication networks 250 (e.g., cellular data, WIFI) to authenticate/authorize the user to user EWOT acknowledgement to the transponder 275 and to access user profiles 265. Direct communication between a wearable computing device 220 and a transponder 275 associated with an EWOT system 225, or a mobile device 210, based on close proximity (which can be determined via close RF communication such as Bluetooth LE) can be supported while either or both devices can also communicate through a network 250 with a remote server 260 for data stored in a user profile 265 in association with the user of the wearable computing device 210 or mobile device 210. Electronic circuitry 225 can include modules such as processors, transmitters, receivers, antennas, etc.

Referring to FIG. 3, a wearable computer 300 in the form of a wristband 220 (as also depicted in FIG. 2) is once again shown. The wearable computer 300 can include at least one sensor 325 operable in association with the electronics 225 integrated within the wristband 220. A sensor 325 integrated with the wearable computing device in the form of a bracelet can measure, track and record data, and the data can also be provided to or accessible by authorized healthcare providers as can be determined by the user profile 265 associated with a user. Features that a wearable computing device 220 can have are listed in the bracket 310 shown in association with the wearable computing device 220. As shown in the listing 310, sensor/module integrated with the wearable computing device 220 can monitor/measure at least one of: pulse rate, oxygenation, temperature, glucose, blood pressure, hemoglobin, activity (e.g., sleep and exercise). The profile can also determine user location for purposes of recording data. A user profile can enable authentication (e.g., via serial number, biometric or passcode) of a user including authentication of healthcare providers obtaining access to a user or the users data.

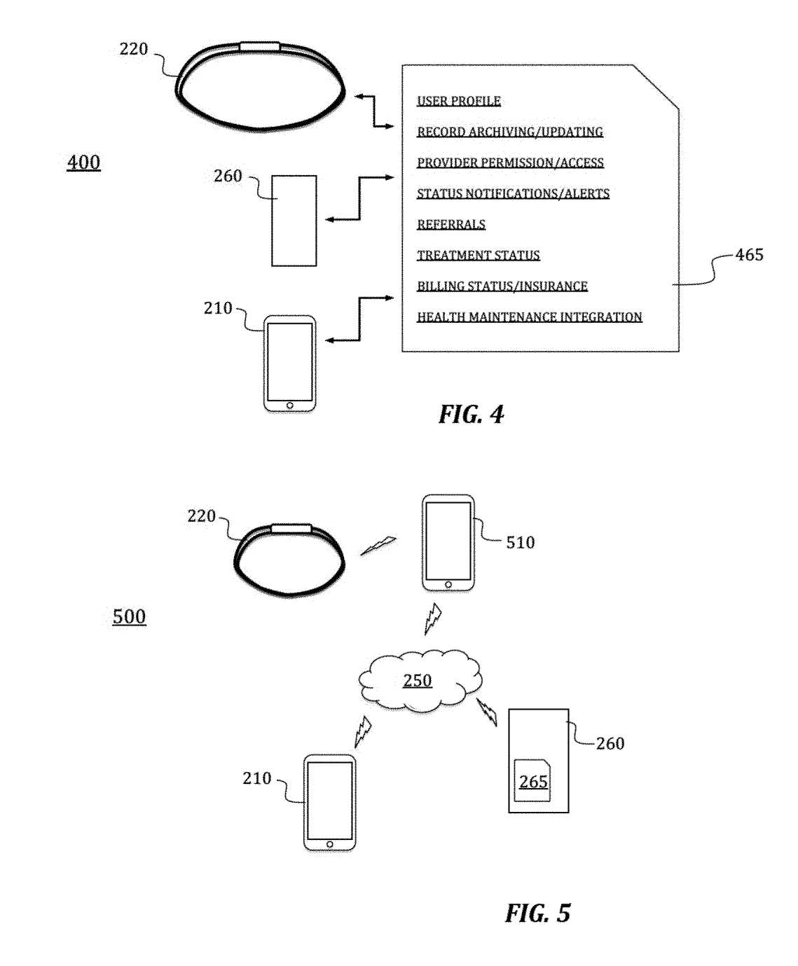

Referring to FIG. 4, a system diagram 400 is depicted with at least three devices (e.g., wristband 220, server 260, and tablet computer/smartphone 210) that can access a user profile 265. As shown in the listing 465, the server 260 can archive and update therapy records, manage access permissions, track user EWOT therapy for the purpose of providing status notifications to interested recipients (e.g., healthcare providers or fitness managers/consultants), enable designation/archiving or referrals, maintain therapy status, manage billing and/or insurance records associated with the user and integrate data into a user's records (e.g., user profile 265) that are associated with health maintenance programs. The user profile 265 can allow for existing service providers to be listed as authorized providers in an electronic template used to authenticate service providers for access to records from the server 260, or receive automated notifications over a data network 250 from the server 260. The template can enable users to electronically authorize providers on their profile 265, remove service providers from their access profile, and enable notifications. Providers that are pending authorization can be listed in a pending list within the profile 265 until either the user or provider electronically acknowledge and authorize inclusion of the provider on the user's access profile so that the provider can access records, receive automated notifications, or both. Automated notifications can be provided for elective procedures, emergency procedures, or both. Other variations of access and provider listing can be designed and provided without departing from the intended scope of the featured aspect of a user access profile 265 as described herein.

Referring to FIG. 5, a network diagram is illustrated to depict how a healthcare provider using a computer 510 and associated with a user using a wearable computing device 220 can receive updated therapy notifications from a server 260 when completed therapy are recorded in the user's profile. A provider 510 (or facility) can provide data for recording in a user's profile records 265 as the user receives EWOT therapy. Any EWOT therapy information can be provided to the notification authorized third parties (e.g., healthcare provider) at their computer 510 via the network 250 in coordination with the server 260 after data is recorded by subsequent providers/facilities providing EWOT therapy to the associated user. The system can synchronize recorded treatment provided to the user and the identity of providers providing EWOT therapy to the user between the at least one server and mobile computing devices associated with the healthcare providers as well as with other providers or third parties authorized by user to receive such data. Updated EWOT therapy records from the at least one server can be provided as a push notification to the computer and/or mobile computing devices associated with the healthcare providers and registered with the user profile.

As mentioned above, a wearable computer 220 can include electronic circuitry, which can further include a wireless communications module (such as cellular, short-range wireless (e.g. Bluetooth, RFID, NFC), or Wi-Fi circuitry) for connection to remote devices (e.g., server 260, transponders 275 and other computing devices 210). It is known that a microprocessor controls all functions (e.g., display, communications, input) in computers. It is also generally known that a wearable computer can further include a rechargeable power source, such as a battery to power the other circuitry.

The communication link is illustrated as a wireless connection; however, wired connections can also be used. For example, the communication link may be a wired serial bus such as a universal serial bus or a parallel bus. A wired connection may be a proprietary connection as well. The communication link can also be a wireless connection using, e.g., Bluetooth™ radio technology, communication protocols described in IEEE 802.xx (including any IEEE 802.11 revisions), Cellular technology (such as GSM, CDMA, UMTS, EVDO, WiMAX, or LTE), or Zigbee™ technology, among other possibilities such as near field communications (NFC) and RFID. The remote device 210 and/or remote server 260 can be accessible via the Internet/Intranet and may include an accompanying smartphone handheld device, a tablet computer, and a computing cluster associated with a particular data services (e.g., electronic medical/health record access and management).

The wearable computing device 220 can also include one or more connection contacts that can be used to connect device 220 to a power source to recharge a battery without removal thereof. Alternative charging functionality can be incorporated into the wearable device, such as electromagnetic recharging, or motion-based electromagnetic charging. Further, the wearable computing device 220 can include a connection port (e.g., USB) that can be used to connect the wearable computing device 210 directly to an external device such as a smartphone or a computer. Port and contacts can be combined as any standardized connection type port such as USB, fire-wire, thunderbolt, or can be provided as specialized connections.

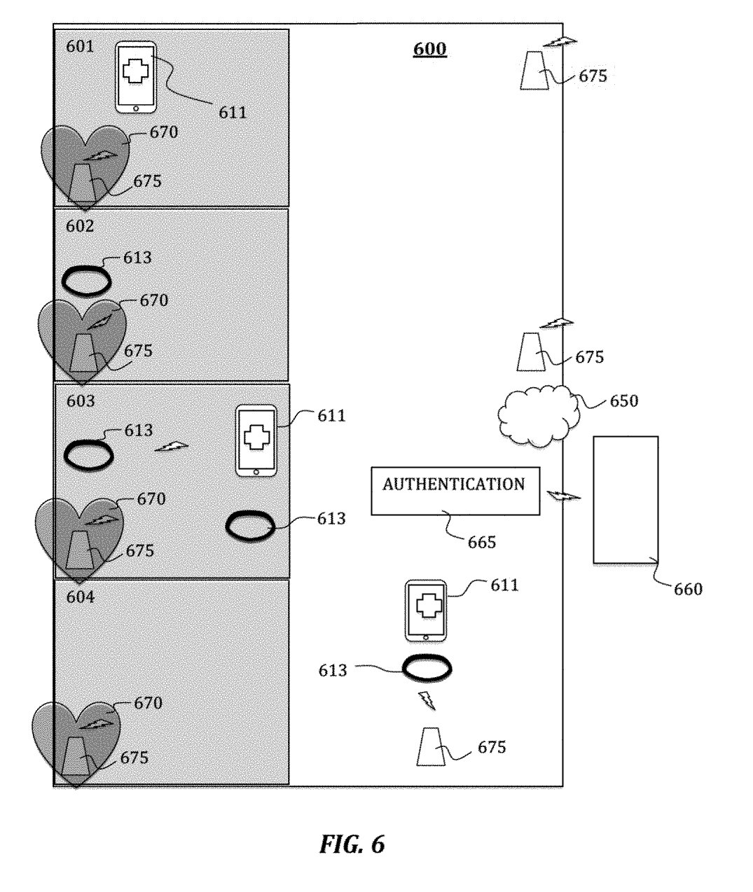

FIG. 6 illustrates an exemplary floor plan of an EWOT therapy facility 600. A facility 600 can take the form of a fitness club or health club and can include therapy areas or rooms 601-604 where a user can access fitness equipment and can meet a provider (e.g., fitness consultant, trainer, or therapist). A wireless network 650 is typically provided within the facility 600, and at least one secure server 660 is typically accessible by users and providers via the wireless network 650 in order to become authenticated 665 with the system and to facilitate any of: use, profile/record retrieval, user association, billing, authorization to, and tracking of the provision of EWOT treatment. In view of features of the present invention, and as shown in room/area 600, a user can use a portable device which can be provided in the form of a handheld device 611 (such as a smartphone, tablet computer) and/or in the form of a wearable device 613, and the handheld device and wearable device can be in communication with teach other, when an associated user accessing EWOT therapy and during therapy. For example, a user wearing a wearable computing device 613 (smart/fitness watch) can communicate wirelessly with an EWOT system 670 located in room/area 602 when in close proximity to the EWOT system 670. A transponder 675 or access point associated with the EWOT system 670 can facilitate communications when near the EWOT system 670, as well as help in determining user locations within the facility 600. In room 603, a healthcare provider can use a wearable computing device 613, similar to the wearable computing device 613 worn by the user, in order to become associated with the user and determine proximity to the healthcare provider to the user. Proximity can be determined by wireless communication (e.g., NFC, RFID, Bluetooth, WiFi, Cellular, etc.). Bluetooth LE (low energy) is a viable communications standard and technology that can support most functions required for this feature, but other standards cannot be ignored.

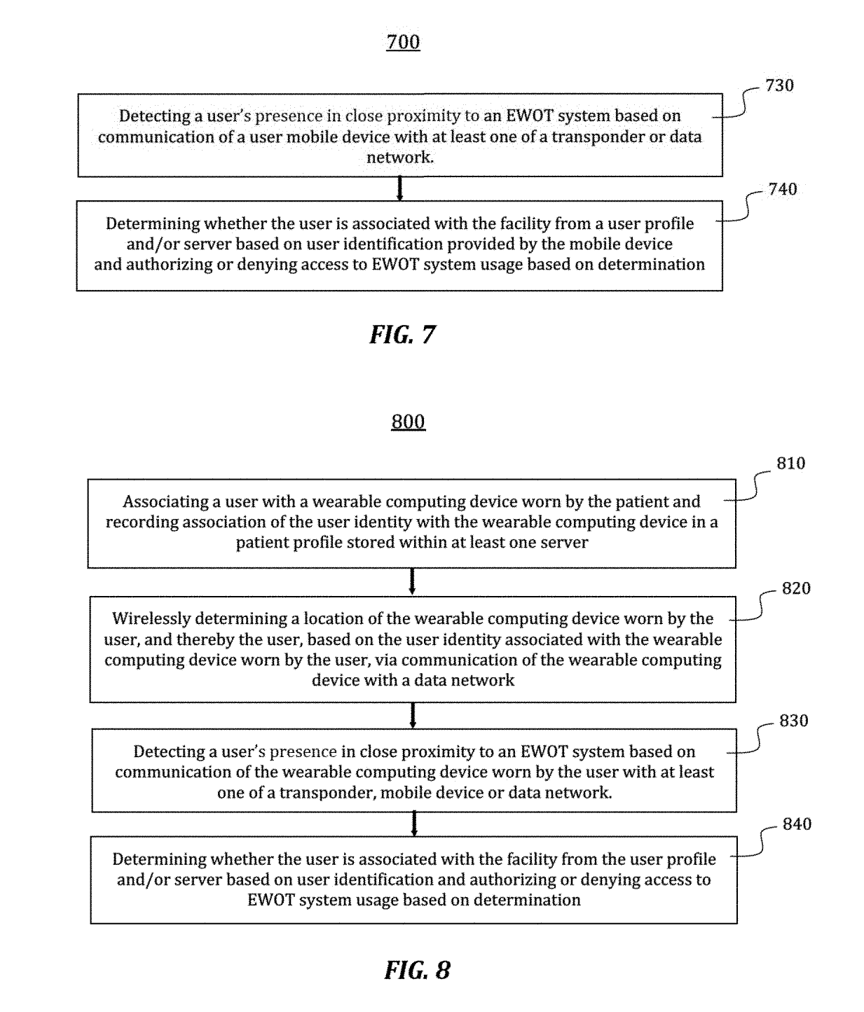

Referring to FIG. 7, a high-level flow chart is shown of a method 700 for associating users with EWOT systems. As shown in block 710, a user's presence in close proximity to an EWOT system is detected based communication of any of a user mobile device with at least one of a transponder or data network. Then, as shown in block 720, it is determined whether a user is associated with the facility from a user profile and/or server based on user identity provided by the mobile device and is authorized or denied access to EWOT therapy based on the determination (access allowed if user access authorization validated).

Referring to FIG. 8, a high-level flow chart is shown of a method 800 for associating users with healthcare providers and providing data and/or services to healthcare providers associated with users, in accordance with features of the present invention. The method is for providing data and/or services to wearable devices. As shown in block 810, a user identity is associated with a wearable computing device worn by a user and the association of the user identity with the wearable computing device is recorded in a user profile stored within at least one server. Then as shown in block 820, location of the wearable computing device worn by the user, and thereby the user, is wirelessly determined based on the user identity associated with the wearable computing device worn by the user via communication of the wearable computing device with at least one of a data network or an EWOT system transponder. As shown in block 830, a user's presence in close proximity to an EWOT system is then detected based on any of the wearable computer, transponder, mobile device, and data network. Then, as shown in block 840, it is determined whether the user is associated with the facility from the user profile and/or server based on user identity and is authorized or denied access to EWOT therapy based on the determination (access allowed if user access authorization validated).

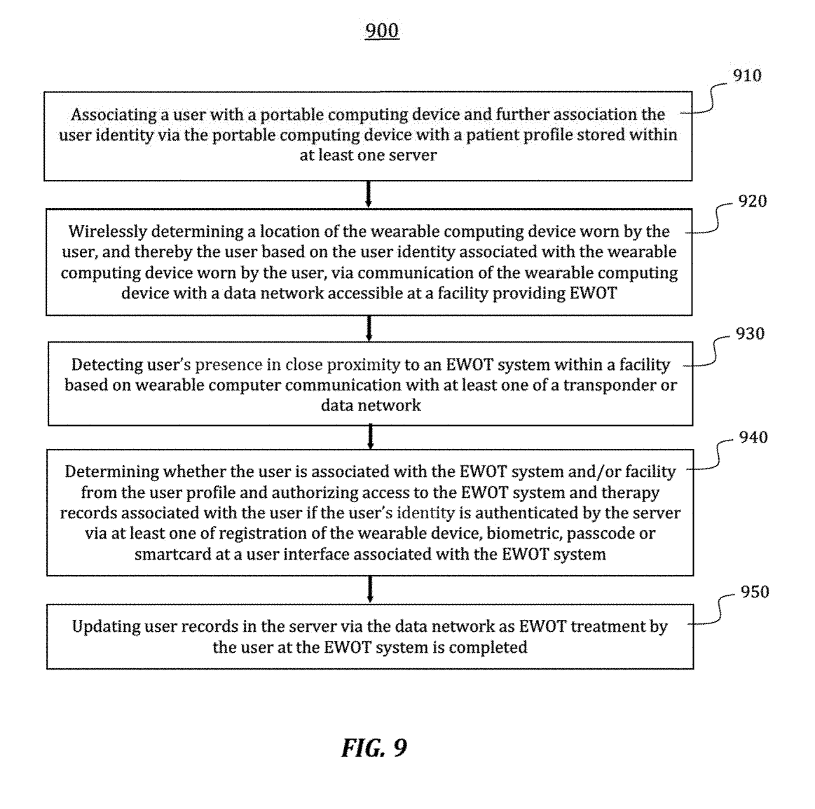

Referring to FIG. 9, a high-level flow chart is shown of a method 900 for associating users with EWOT systems and facilities and providing data and/or services to authenticated users, in accordance with features of the present invention. As shown in block 910, a user is associated with a portable computing device and the user identity via the portable computing device is associated with a user profile stored within a least one remote server. Then as shown in block 920, wireless determining the location of the portable computing device worn by the user, and thereby the user, based on the user identity associated with the portable computing device worn by the user via communication of the portable computing device with a data network accessible at a facility providing EWOT. As shown in block 930, detecting the user's presence in close proximity to an EWOT system within the facility based on portable computer communication with at least one of a transponder or data network at the facility. Then, as shown in block 940, it is determined whether the user is associated with the EWOT system and/or facility from the user profile and authorizing access to the EWOT system and therapy records associated with the user if the user's identity is authenticated by the server via at least one of registration of the wearable device, biometric, passcode or smartcard at a user interface associated with the EWOT system. Then as shown in block 950, user records are updated in the server view the data network as EWOT treatment by the user at the EWOT system if completed.

The aforementioned authorization steps shown in blocks 840 and 940 can further include a step or logical operation for determining the identity of the user and providing the user access to the data and/or the services based on the identity of the user. Examples of data are, for example, coupons, advertising information, video, video clips, replays, statistics, information, text, voice, etc. Examples of services are, for example, tour guides (self-guided tours), providing historical information with respect to a point of interest, providing entertainment information (e.g., voice, text, etc.) to fans at a sporting or concert event, providing medical data and user monitoring during, for example, surgery, treatment, and recovery. Other examples of services include providing assistance to drivers to prevent fatigue and auto accidents, and directional and navigational information to drivers.

A biometric scanner can be integrated with an optical and image-processing system associated with the EWOT system in the form of a user interface, or via the wearable device, and/or can be implemented as an “app” that enables the wearable device to perform biometric scanning (recognition) operations. The wearable device can be implemented as head gear worn by a user. Examples of such headgear include, for example, eyeglasses or a hardware system configured in the form of virtual reality gaming goggles worn by the user.

In accordance with another feature, the aforementioned at least one biometric may be, for example, a retinal scan gathered through optics integrated with the wearable device. In yet another embodiment, the at least one biometric can include at least one other biometric gathered through the wearable device. The wearable device may be implemented as data enabled eyewear. Additionally, in some embodiments, the aforementioned authenticating steps shown can be facilitated by a remote server (e.g., a server or group of servers). The data and/or the services can be accessed and retrieved from such a remote server based on the identity of the user. As shown in blocks 730 and 830, a user's presence in close proximity to EWOT system can be detected (e.g., via Bluetooth, RFID, NFC, etc.) based on a user's portable computing device carried or wearable computing device worn by the user.

Authentication can be provided for wirelessly communicating data and/or services between the wearable device and at least one transponder out of a plurality of transponders and dispersed with a facility. The at least one transponder is preferably within wireless communications range (e.g., Bluetooth LERFID, NFC) of the user's mobile/wearable computing device. One example of a transponder that can be implemented in accordance with one or more embodiments is the “iBeacon.” iBeacon is the trademark for the proximity system that Apple Inc. has referred to as “a new class of low-powered, low-cost Bluetooth transmitters that can notify nearby iOS devices of their presence.” The technology enables an iOS device or other hardware to send push notifications to iOS devices in close proximity. Devices running the Android operating system, for example, can receive iBeacon advertisements but cannot emit iBeacon advertisements (i.e., central role only). Currently, the iBeacon operates on Bluetooth Low Energy (BLE), also known as Bluetooth Smart. BLE can also be found on Bluetooth 4.0 devices that support dual mode. iBeacon uses Bluetooth low energy proximity sensing to transmit a universally unique identifier capable of being picked up by a compatible app or operating system that can be turned into a physical location or trigger an action on the device.

Note that in an embodiment, a step or logical operation can be provided for authenticating an identify of the healthcare provider prior to authorizing access to the healthcare records, wherein authentication comprises: at least one of acquiring a biometric; and acquiring entry of a passcode from the healthcare provider; and an RFID tag and/or NFC-enabled credentialed smartcard or worn bracelet that is challenged throughout a healthcare facility (e.g., upon entry into a user's room, wherein a user awaits treatment).

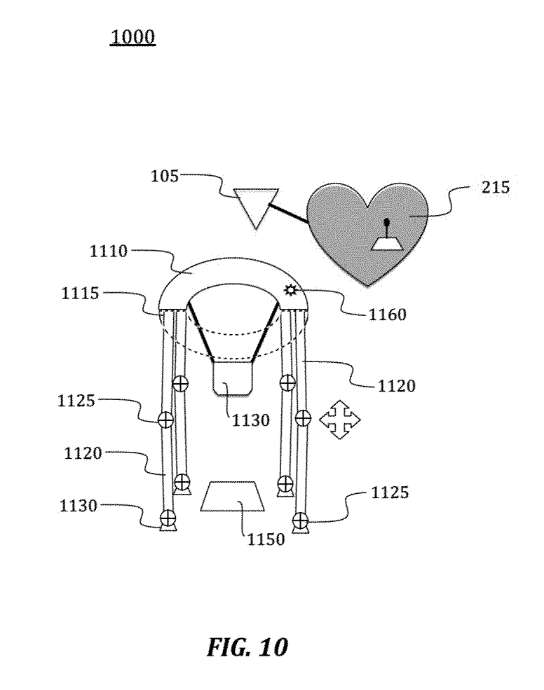

Referring to FIG. 10, illustrated is a safety harness system 1000 that can be adapted for use with exercise equipment 1150 during EWOT therapy. The safety harness system 1000 includes a circular padded rail 1110/1115 that can encircle the waist or torso of a user underneath the user's arms. The harness system can includes hydraulically controlled legs 1120 that enable the harness 1000 to be manipulated/moved by the patient/user when secured to the user and while the user is utilizing exercise equipment 1150. The hydraulics 1125 can slow/control/cause the bending action of each leg 1120 while a user moves about exercise equipment 1150. The harness system 1000 can include any number of sensors 1160 and/or transponder therein to record user activity, authorize use of the system, and report use to a remote server as taught herein. The primary purpose of the harness system 1000, however, is security over the user while lessening the physical requirements on a therapist during EWOT therapy. The safety harness system 1000 can also include a seat area 1130 and straps for further securing a patient/user within the harness 1000. The about half of circular portion of the harness 1115 (showed in dashed lines) can be partially opened (e.g., hinged and lockable) to enable a user to enter the padded circular portion 1110/1115 of the harness 1000. The opening portion of the harness can then be closed and secured over a user. An EWOT system 215 can be co-located next to the safety harness system 1000 and exercise equipment 1150, where a user can access enhanced oxygen from a mask 105.

It should be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A method for providing user access to EWOT systems, said method comprising:
wirelessly detecting a presence of a user mobile device in close proximity to an EWOT system in a facility;
determining whether the user is authorized to use the EWOT system based on association of the user via the mobile device with a user profile accessible via a data network from a remote server; and
authorizing the user's access to the EWOT system when and if confirmed by the remote server.

2. The method of claim 1, wherein the step of determining whether the user is authorized includes the step of authenticating an identity of the user with credentials including at least one of: a biometric acquired from the user; a passcode accepted by the user in a user interface; acquiring a signal from an RFID tag and/or NFC-enabled credentialed device carried by the user.

3. The method of claim 1, wherein the mobile device carried by the user comprises a wearable computing device worn by the user.

4. The method of claim 1, including authenticating the identity of the user occurs prior to authorizing access to the EWOT system, wherein authentication is provided by at least one of: acquiring a biometric; and acquiring entry of a passcode from the user on the user interface.

5. The method of claim 4, further comprising wherein said authenticating is facilitated over the data network by the at least one remote server.

6. The method of claim 4, wherein data and/or services are accessed by the healthcare provider based on the authentication and is retrieved over the data network from the at least one remote server.

7. The method of claim 3, further comprising associating the wearable computing device worn by the healthcare provider with a wireless hand held computer, wherein the wireless hand held computer includes a display screen and user interface for the healthcare provider to review, create, and manage records associated with the user.

8. The method of claim 1, wherein the step of wirelessly determining the location of the wearable computing device worn by the user is facilitated by at least one transponder out of a plurality of transponders dispersed throughout the facility.

9. A system for authorizing user access to an exercise with oxygen therapy (EWOT), comprising:
an EWOT system including an oxygen source including a connection for an oxygen mask and collocated with exercise equipment; and
a user interface also collocated with the exercise equipment enabling a user to interact with the user interface physically or via a portable device associated with the user and obtain authorization to use the EWOT system.

10. A system of claim 9, comprising:
a fitness and health facility containing at least one of the EWOT system and an associated user interface collocated with a plurality of exercise equipment also located within the fitness and health facility.

11. The system of claim 9, wherein the authorization can be obtained by at least one of: entry of a passcode, entry of a biometric, synchronization with a smart card, swiping a magnetic card, and wirelessly connecting via a mobile device.

12. The system of claim 10, wherein the fitness and health facility further includes a data network including a plurality of transponders deployed throughout the fitness and health facility, wherein the transponders enable determination of wearable device location the data network facilitates user authentication and access to records.

13. The system of claim 10, wherein user association with an EWOT system is determined from a user profile stored on the at least one server when a user wearing a wearable device is determined by at least one transponder to be in close proximity to an EWOT system, and the user is authorized access to at least one of the EWOT system and data associated with the user based on determination.

14. The system of claim 9, wherein the user interface includes a biometric scanner for authenticating healthcare provider identity, said biometric scanner further comprising at least one of: an iris recognition scanner, a voice recognition scanner, a fingerprint recognition device, an ear acoustical scanner for biometric identification using acoustic properties of an ear canal.

15. A method for providing security to a user of exercise equipment while the user moves on or about the exercise equipment, said method comprising:
providing a safety harness system in association with exercise equipment, the safety harness system further including a circular padded rail that can encircle the waist or torso of a user underneath the user's arms, hydraulically controlled legs that enable the safety harness system to be manipulated/moved under hydraulic control when the padded rail is secured to the user and while the user is moving on exercise equipment, wherein hydraulics integrated with the hydraulically controlled legs slow/control bending action of the legs while a user moves about the exercise equipment, wherein the safety harness system traverses the workout equipment;
accepting a user into the circular padded rail and securing the user within the padded rail; and
wherein the safety harness system moves under hydraulic control as the user is moving on the exercise equipment.

16. The method of claim 15, further comprising the steps of:
wirelessly detecting a presence of a user mobile device in close proximity to an EWOT system associated with the exercise equipment;
determining whether the user is authorized to use the EWOT system based on association of the user via a mobile device in further association with a user profile accessible from at least one of the mobile device or via a remote server through a data network via the mobile device; and
authorizing the user's access to the EWOT system when and if confirmed by the remote server.

17. The method of claim 16, wherein the step of determining whether the user is authorized includes the step of authenticating an identity of the user with credential including at least one of: a biometric acquired from the user; a passcode accepted by the user in a user interface; acquiring a signal from an RFID tag, and/or NFC-enabled credentialed device carried by the user.

18. The method of claim 16, wherein the mobile device carried by the user comprises a wearable computing device worn by the user.

19. The method of claim 18, including authenticating the identity of the user occurs prior to authorizing access to the EWOT system, wherein authentication is provided by at least one of: acquiring a biometric; and acquiring entry of a passcode from the user on the user interface.

20. The method of claim 19, further comprising wherein said authenticating is facilitated over the data network by the at least one remote server.
